(19) United States
(12) Patent Application Publication
(10) Pub. No.: US 2016/0120664 A1
(43) Pub. Date: May 5, 2016

(54) BREATH AND HEAD TILT CONTROLLED PROSTHETIC LIMB

(71) Applicant: Marin Renner SCHULTZ, Alberta (CA)

(72) Inventor: Marin Renner SCHULTZ, Alberta (CA)

(21) Appl. No.: 14/925,878

(22) Filed: Oct. 28, 2015

Related U.S. Application Data
(60) Provisional application No. 62/073,736, filed on Oct. 31, 2014.

(51) Int. Cl. A61F 2/68; A61F 2/58

(52) U.S. Cl. CPC A61F 2/68; A61F 2/583; A61F 2002/704

(57) ABSTRACT

In alternative embodiments, provided is an internally powered prosthetic limb and method for controlling same, offering hands-free control of a prosthetic limb. The internally powered prosthetic limb controlled by an input control device, comprising: a breath inlet configured to receive air pressure exerted from a user's mouth to be converted into a first electronic signal, wherein the first electronic signal is proportional to the exerted pressure; a motion sensor configured to sense tilting of the user's head and operable to convert the user's head tilting motion into a second electronic signal, wherein the second electronic signal is proportional to the user's head tilting motion; and a processor in communication with the breath inlet and the motion sensor, the processor operable to convert the exerted air pressure into the first electronic signal and further process the first electronic signal into first positional data for transmission to a plurality of digit-actuators in the prosthetic limb, the processor further operable to process the second electronic signal into second positional data for transmission to a wrist-actuator in the prosthetic limb; wherein the plurality of digit-actuators in the prosthetic limb can be controllably actuated in proportion to the air pressure exerted by the user, and the wrist-actuator can be controllably actuated to rotate in proportion to the head tilting motion of the user.

### RELATED APPLICATIONS

This U.S. Utility patent application claims the benefit of priority under 35 U.S.C. §119(e) of U.S. Provisional Application No. 62/073,736, filed Oct. 31, 2014. The aforementioned application is expressly incorporated herein by reference in its entirety and for all purposes.

### TECHNICAL FIELD

The present invention relates to the field of functional limb prosthetics and, in particular, to an internally powered prosthetic limb that is controlled by the user's breath and head tilt.

### BACKGROUND OF THE INVENTION

A functional limb prosthetic replaces the function of an amputated or congenitally malformed or missing limb in order to offer functionality to the individual. Technological advances have resulted in vast improvements in the functionality of such prosthetics. Where functional limb prosthetics were once limited to being mechanically controlled through cables and harnesses strapped to the individual, internally powered limb prosthetics that use a battery and an electronic system to control movement have been developed.

At the forefront of internally powered prosthetic technology is the myoelectrically controlled prosthetic which uses electronic sensors to detect minute muscle, nerve, and EMG activity. This muscle activity is then translated into information that can be used by electric motors to control the movements of the prosthetic limb. In this way, the prosthetic limb can be controllably maneuvered much like a natural limb, according to the neural stimulus of the user.

Further developments made in prosthetic control have also explored the possibility of a brain machine interface, wherein the prosthetic limbs would be directly actuated and controlled by brain signals (e.g., intracranial electroencephalography or iEEG and traditional EEG signals).

United States Patent Publication No. 2006/0167564 describes a biological interface apparatus that detects and processes multicellular signals from the central or peripheral nervous system of an individual and transmits these signals to a joint movement device such as a prosthetic limb to afford the patient voluntary control of the prosthetic. Such control systems are complex and can be invasive, requiring implantation of the apparatus into the patient's body. As well, these types of control systems are expensive and cost prohibitive to most of the population. Accordingly, access to many forms of existing powered prosthetic limbs is limited.

There continues to be a need, therefore, for an alternative powered prosthetic limb that offers both reliable user-control and robust functionality at a non-prohibitive cost.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

Disclosed herein are exemplary embodiments pertaining to a breath and head-tilt controlled prosthetic limb. In accordance with one aspect, there is described an input control device for a power-driven prosthetic limb, comprising: a breath inlet configured to receive air pressure exerted from a user's mouth to be converted into a first electronic signal, wherein the first electronic signal is proportional to the exerted pressure; a motion sensor configured to sense tilting of the user's head and operable to convert the user's head tilting motion into a second electronic signal, wherein the second electronic signal is proportional to the user's head tilting motion; and a processor in communication with the breath inlet and the motion sensor, the processor operable to convert the exerted air pressure into the first electronic signal and further process the first electronic signal into first positional data for transmission to a plurality of digit-actuators in the prosthetic limb, the processor further operable to process the second electronic signal into second positional data for transmission to a wrist-actuator motor in the prosthetic limb; wherein the plurality of digit-actuators in the prosthetic limb can be controllably actuated in proportion to the air pressure exerted by the user, and the wrist-actuator can be controllably actuated to rotate in proportion to the head tilting motion of the user.

According to certain embodiments of the input control device, the first positional data actuates the plurality of digit-actuators into a closed position. According to other embodiments, the first positional data corresponds to customized pre-programmed digit-actuator control impulses to actuate pre-programmed movement combinations.

According to further embodiments, the input control device described herein further comprises a feedback control loop for controlling an upper limit of actuating the plurality of digit-actuators. In particular embodiments, the feedback control loop comprises one or more pressure sensors in each of the plurality of digits, each pressure sensor configured to sense pressure exerted by the respective digit against an object and operable to convert the exerted pressure into a feedback electronic signal proportional to the exerted pressure, wherein the feedback electronic signal is transmitted to the processor to halt actuation of the plurality of digit-actuators when the upper limit is reached.

In accordance with another aspect, there is described a powered prosthetic limb, comprising: a first input control comprising a breath inlet configured to receive air pressure exerted from a user's mouth to be converted into a first electronic signal, wherein the first electronic signal is proportional to the exerted pressure; a second input control comprising a motion sensor configured to sense tilting of the user's head and operable to convert the user's head tilting motion into a second electronic signal, wherein the second electronic signal is proportional to the user's head tilting motion; a prosthetic limb comprising a plurality of digit-actuators and a wrist-actuator, each digit-actuator attached to a respective artificial tendon or gear system, wherein the respective artificial tendon or gear system can be retracted or activated by the respective digit-actuator; and a processor housed in the prosthetic limb in communication with the first and second input control device, the processor operable to convert the exerted air pressure into the first electronic signal and further process the first electronic signal into first positional data for transmission to the plurality of digit-actuators in the prosthetic limb, the processor further operable to process the second electronic signal into second positional data for transmission to the wrist-actuator in the prosthetic limb; wherein the plurality of digit-actuators in the prosthetic limb can be controllably actuated in proportion to the air pressure exerted by the user, and the wrist-actuator can be controllably actuated to rotate in proportion to the head tilting motion of the user.

According to embodiments of the powered prosthetic limb, the first positional data actuates the plurality of digit-actuators into a closed position. According to other embodiments, the first positional data corresponds to customized pre-programmed digit-actuator control impulses to actuate pre-programmed movement combinations.

According to further embodiments, the prosthetic limb described herein, further comprises a feedback control loop for controlling an upper limit of actuating the plurality of digit-actuators. In particular embodiments, the feedback control loop comprises one or more pressure sensors in each of the plurality of digits, each pressure sensor configured to sense pressure exerted by the respective digit against an object and operable to convert the exerted pressure into a feedback electronic signal proportional to the exerted pressure, wherein the feedback electronic signal is transmitted to the processor to halt actuation of the plurality of digit-actuators when the upper limit is reached.

In accordance with a further aspect, there is described a method of controlling a prosthetic limb, comprising: receiving air pressure from a user and converting the air pressure to a first electronic signal, wherein the first electronic signal is proportional to the air pressure; sensing a tilting motion of the user's head and converting the tilting motion into a second electronic signal, wherein the second electronic signal is proportional to the user's head tilting motion; processing the first electronic signal into first positional data for a plurality of digit-actuators in the prosthetic limb and processing the second electronic signal into second positional data for a wrist-actuator in the prosthetic limb; and transmitting the first positional data to the plurality of digit-actuators, and transmitting the second positional data to the wrist-actuator; wherein the plurality of digit-actuators in the prosthetic limb can be controllably actuated in proportion to the air pressure exerted by the user, and the wrist-actuator can be controllably actuated to rotate in proportion to the head tilting motion of the user.

According to embodiments of the method, the first positional data actuates the plurality of digit-actuators into a closed position. According to further embodiments, the first positional data corresponds to customized pre-programmed digit-actuator control impulses to actuate pre-programmed movement combinations.

According to other embodiments, the method further comprises: sensing pressure exerted by one or more digits against an object and converting the exerted pressure into a feedback electronic signal proportional to the exerted pressure; and transmitting the feedback electronic signal to the processor effecting an upper limit for actuation of the plurality of digit-actuators; wherein actuation of the plurality of digit-actuators is halted when the upper limit is reached. According to particular embodiments, the processor compares the feedback electronic signal with preset pressure signal limits to effect the upper limit. According to further embodiments, one or more digit pressure sensors is used to sense the pressure exerted by one or more digit-actuators against an object. In such embodiments, the one or more digit pressure sensors is a squeeze sensor, a pressure sensitive wafer, or a force sensitive resistor.

According to certain embodiments, the air pressure received from the user is positive air pressure created by the user exhaling air. According to other embodiments, the air pressure is negative air pressure created by the user inhaling air. According to further embodiments, the air pressure is a combination of positive and negative air pressure created by the user exhaling and inhaling air in various durations and combinations.

### BRIEF DESCRIPTION OF THE DRAWINGS

These and other features of the invention will become more apparent in the following detailed description in which reference is made to the appended drawings.

FIG. 1 is a perspective view of an input control device for a power-driven prosthetic limb, according to embodiments of the present disclosure;

FIG. 2 is a perspective view of a powered prosthetic limb controllable by the input control device shown in FIG. 1, according to embodiments of the present disclosure;

FIG. 3 is an electrical schematic diagram showing the basic components constituting the control circuitry of the input control device and the prosthetic limb shown in FIG. 2, according to embodiments of the present disclosure; and

FIG. 4 is a flow chart illustrating a method of operation for a powered prosthetic limb, according to embodiments of the present disclosure.

### DETAILED DESCRIPTION OF THE INVENTION

The powered prosthetic limb according to embodiments of the present disclosure offers a cost-effective approach to hands-free control of a prosthetic limb. Specifically, the prosthetic limb of the present disclosure allows the user to control at least two parameters of a prosthetic limb (for example, finger movement and wrist rotation) using a single control device measuring breath and head tilt, respectively. According to certain embodiments, the prosthetic limb includes an input control device that comprises a breath inlet configured to receive air pressure exerted from a user's mouth. The air pressure is converted to positioning information transmitted to actuate a plurality of digits in the prosthetic limb into a closed position in proportion to the air pressure exerted by the user. Hands-free control over the gripping action of a prosthetic hand is offered to the user. According to embodiments, the gripping action of the prosthetic hand can be readily controlled by the user simply by inhaling or exhaling into the breath-pressure sensing tube. According to embodiments, pre-programmed hand positions, gestures, or movement combinations can be activated by combinations of positive and negative air pressures exerted by the user into the air inlet. This allows the user to easily make different, complex hand gestures with simple pressure-change combinations.

A second motion can be controllably actuated according to embodiments of the present disclosure. In particular, the input control device can further comprise a motion sensor configured to sense tilting of the user's head. The head tilting motion is converted to positioning information transmitted to actuate a wrist-actuator in the prosthetic limb to rotate in proportion to the head tilting motion of the user. According to certain embodiments, rotation of the wrist is proportional to the head tilting motion of the user. In other embodiments, the wrist is rotated in the same direction as the user's head tilting motion. In this way, control over the wrist rotation of the prosthetic arm is intuitive and easy to operate.

According to embodiments of the present disclosure, the input control device is conveniently adapted to be wearable by the user. For example, the input control device can be implanted within a headset or an earpiece to be worn on the user's head. In this way, the input control device can be stably secured into position for easy operation in an unobstructive manner. The prosthetic limb, according to the embodiments described herein, comprises all the electronics required for operation. In this way, the prosthetic limb of the present disclosure offers a generally self-contained system that is aesthetically pleasing and compact for the user. According to further embodiments, the input control device can be miniaturized and can communicate wirelessly with the prosthetic limb. Although the input control device has been described as a headset or an earpiece worn on the user's head, it will be understood that the input control device can take other forms for securely positioning the device on the user for independent operation by the user.

### Definitions

Unless defined otherwise, all technical and scientific terms used herein have the same meaning as commonly understood by one of ordinary skill in the art to which this invention belongs.

As used herein, the term “head tilt” refers to head flexion along all axes including, without limitation, lateral flexion of the neck (from side to side) along the sagittal axis in the frontal plane.

As used herein, the term “about” refers to an approximately +/−10% variation from a given value. It is to be understood that such a variation is always included in any given value provided herein, whether or not it is specifically referred to.

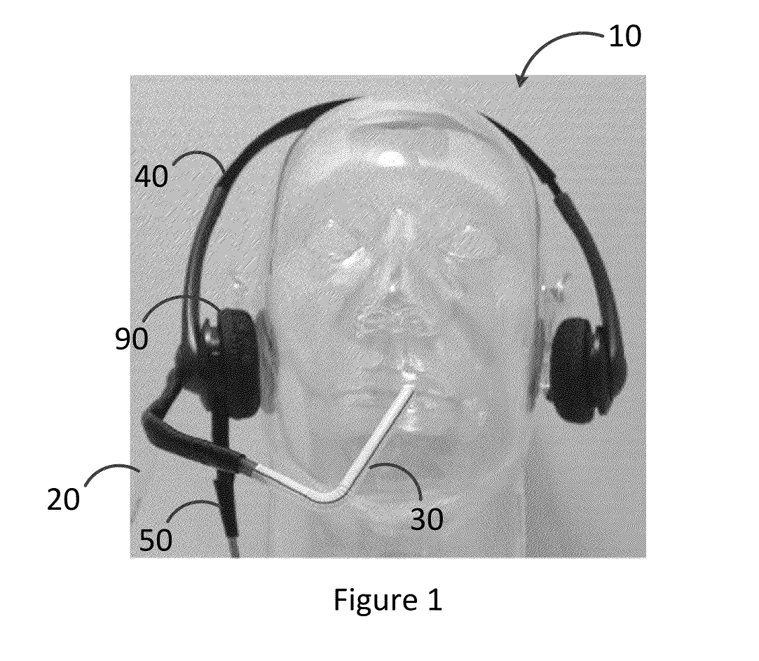

Embodiments of the present disclosure will now be described by reference to FIGS. 1 to 4, which show representations of the powered prosthetic limb according to the present disclosure.

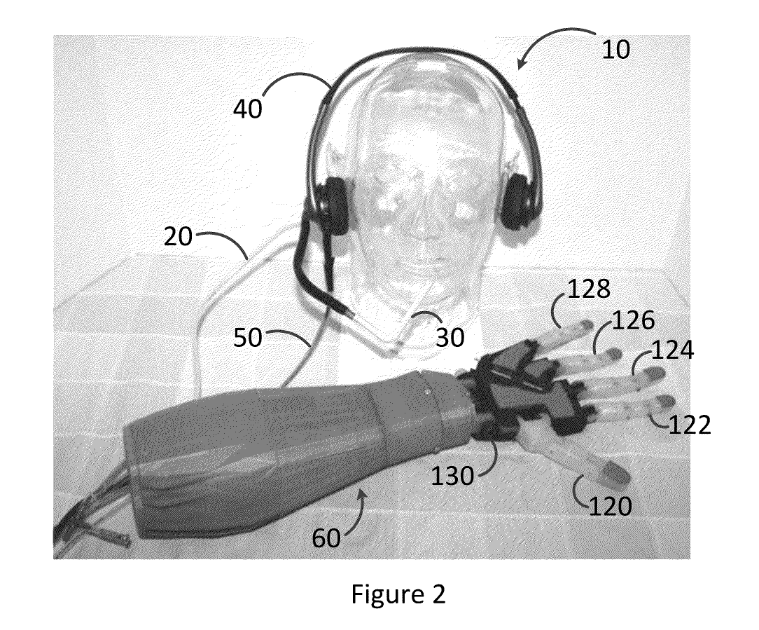

An input control device constructed in accordance with an exemplary embodiment of the present disclosure is shown in FIG. 1 and is designated generally as 10. The input control device 10 is in operable communication with a power-driven prosthetic limb 60, for example a prosthetic arm 60 according to the embodiments described herein and shown in FIG. 2.

### Breath Pressure Control—First Movement

The input control device 10 comprises a breath inlet 30 configured to receive air pressure exerted from a user's mouth and conveyed to a breath-pressure sensing tubing 20. In accordance with other embodiments, the breath pressure sensor may reside inside the earpiece 90 and will not require additional tubing. According to certain embodiments, the breath inlet 30 may be manufactured from flexible plastic. According to other embodiments, metal wire can be embedded in the plastic, such that the breath inlet 30 can be bent and maintained in a desired shape. The breath inlet 30 may be manufactured from other materials or combinations of materials that allow flexible and adjustable connection of the breath inlet 30 with the headset 40, such as rubber hosing with embedded metal wire, or plastic shaped with a bend for positioning into the mouth of the user. Most importantly, the critical property of the breath inlet 30 is to provide the user with the ability to access and/or retain the breath inlet 30 in his mouth in a comfortable position for possibly extended periods of time. According to certain embodiments, the breath inlet 30 can be removed and replaced as wear and build-up of contaminants dictate.

According to certain embodiments, the breath inlet 30 may further comprise a protective tip (not shown) to improve the comfort of use to the user. The protective tip may be a plastic attachment fastened at the mouth receiving end of the breath inlet 30. The protective tip may be manufactured from plastic or other materials, such as certain metals provided that they can be formed to the proper size and shape and that they can be cleaned and sterilized. Thus, the protective tip can be removed and replaced as wear and build-up of contaminants dictate.

As shown in FIGS. 1 and 2, the input control device 10 can be generally worn as a headset 40. Any headset of a design capable of securing the input control device 10 in position on the head of a user can be used and is contemplated by the present disclosure. According to certain embodiments, the input control device 10 can take the form of an earpiece that can be attached to the user's ear with the breath inlet 30, in such an embodiment, comprising piping to the mouth.

The breath inlet 30 may be removeably coupled to the headset 40, as shown in FIGS. 1 and 2. For example, the breath inlet 30 may be sized at the end opposite the breath receiving end to allow the breath inlet 30 to slide into the input port of the headset 40. Other means of fastening, such as threaded mating, are possible that hold the breath inlet 30 in the input port of the headset 40 while still allowing the breath inlet 30 to be removed and/or replaced. The breath-pressure sensor tubing 20 provides air pressure communication to a pressure sensor (not shown) housed within the prosthetic limb 60, according to certain embodiments, and operable to convert the exerted air pressure into a first electronic signal, wherein the first electronic signal is proportional to the exerted pressure. According to alternative embodiments, the pressure sensor can be located within the headset 40, the breath inlet 30 or the breath-pressure sensor tubing 20.

The pressure sensor may be coupled with the tubing 20 and is operable to receive air pressure from the user through the tubing 20. According to certain embodiments, the air pressure may be positive, and generated by the user exhaling. According to other embodiments, the air pressure may be negative, and generated by the user inhaling. According to further embodiments, the air pressure may be a combination of patterned positive and negative pressure changes generated by the user exhaling and inhaling respectively. To convert the air pressure into an electronic signal, the pressure sensor may be a transducing element. The pressure sensor is configured to detect a change in positive and/or negative pressure and subsequently output the appropriate electronic signal. For example, a pressure sensor able to sense a range of pressure from 0 to 10 kPa (0 to 1.4 psi) is configured to measure pressure from −5 kPa to +5 kPa (−0.7 psi to +0.7 psi). In this configuration, the pressure sensor may detect both positive and/or negative pressure. The pressure sensor may include transducing elements such as strain gauges, piezoresistive semiconductors, and micro-electro-mechanical systems (MEMs).

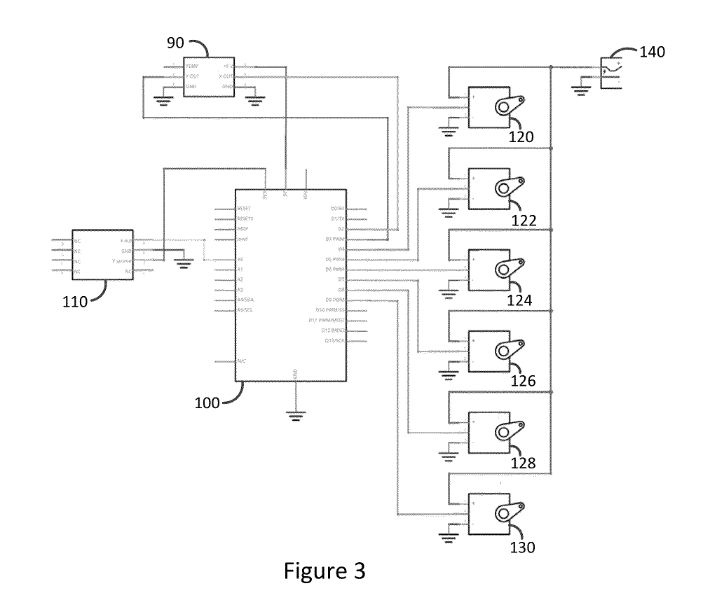

An embodiment of the electronic control circuitry of the input control device 10 and the prosthetic limb 60 are illustrated in electronic schematic diagram form in FIG. 3. The electronic signal output of the pressure sensor 110 is converted into positioning data for the prosthetic limb 60. The output from the pressure sensor 110 is typically an analog voltage which typically must be converted and formatted to a standard protocol in order to be transmitted and operable for the prosthetic limb 60. The analog output of the pressure sensor 110, according to embodiments, is converted to a digital format by an analog to digital converter (ADC). According to certain embodiments, an amplifier, and/or other mediating/filtering circuit known in the art, between the pressure sensor 110 and the converter, may be used to condition the pressure sensor 110 output signal.

The processor 100 generally translates the pressures received from the user, through the pressure sensor 110, the amplifier, and any other mediating/filtering circuit or data converter, into positional data for actuating motors 120, 122, 124, 126, 128, for example servo motors or micro-stepper motors, to move the fingers of the prosthetic limb 120, 122, 124, 126, 128 in a first movement.

In various embodiments, data is generated by the pressure sensor 110, the amplifier, and any other mediating/filtering circuit or data converter, and transmitted to the processor 100, then further transmitted by the processor 100 to the respective motors 120, 122, 124, 126, 128. Transmission of the data at both stages can be achieved by a standard data cable known in the art. For example, the data cable may consist of simple stranded copper wire, or it may be a standard USB cable, although other compatible data cables are possible. Generally, other means of data communication are also possible. In some embodiments, it is possible that a fiber optic cable may be used for the data cable providing that the data output is converted from electrical data to optical data by an optical transmitter. In other embodiments, data may be communicated wirelessly providing that the data output is transmitted by a radio frequency (RF) transmitter.

According to preferred embodiments, the prosthetic limb 60 is a prosthetic arm 60 as illustrated in FIG. 2 that comprises a plurality of digit-actuators in operable communication with the processor. According to certain embodiments, the prosthetic arm 60 comprises up to five digit-actuators 120, 122, 124, 126, 128, for example finger-servo motors, to allow the fingers of the prosthetic arm 60 to be controllably actuated into a closed position in proportion to the air pressure exerted by the user. To actuate closing of each finger, each finger-servo motor 120, 122, 124, 126, 128 can be attached to a respective artificial tendon or gear system, wherein the respective artificial tendon or gear system can be refracted or activated by the respective finger-servo motor to the closed position.

According to embodiments of the present disclosure, the digit-actuators can be independently actuated to offer the user control over one or more fingers at a time. According to other embodiments, all of the digit-actuators are actuated simultaneously in order to effect the gripping motion. According to further embodiments, gear-driven, micro stepper-motors could be positioned in every finger joint to give more grip strength to the hand. According to further embodiments, the first positional data can be customized with pre-programmed digit-actuator control impulses corresponding to a pattern of air pressure changes created by the user, for example. In this way, the digit-actuators may be actuated to perform pre-programmed movement combinations that further expand the scope of movements made possible by the instant prosthetic limb.

In certain embodiments, the finger tips or pads of the prosthetic can further include pressure sensors to allow the grip strength to be controlled. In such an embodiment, for example, a quick pressure change in the breath sensor could be used to effect hand closure until finger sensor limits are reached. According to such embodiments, the prosthetic limb can comprise a feedback control loop for controlling the upper limit of actuating the plurality of digit-actuators. Such a feedback control loop can comprise one or more pressure sensors in each of the plurality of digits, each pressure sensor configured to sense pressure exerted by the respective digit actuator against an object and operable to convert the exerted pressure into a feedback electronic signal proportional to the exerted pressure, wherein the feedback electronic signal is transmitted to the processor to halt actuation of the plurality of digit-actuators when the upper limit is reached. According to certain embodiments, the processor can be configured to compare the feedback electronic signal to preset pressure signal limits, for example, to effect an upper limit for actuation of the plurality of digit-actuators, wherein actuation of the plurality of digit-actuators is halted when the upper limit is reached.

According to further embodiments, data for haptic feedback could also be provided to the user giving the user the ability to “feel” the held object. According to certain embodiments, for example, a proportionally vibrating motor or other active device can be used to provide such haptic feedback. Further embodiments may take advantage of cutting edge nerve-induction techniques for haptic feedback.

Pressure sensors known in the art can be used in the digit of the prosthetic limb to sense the pressure exerted by one or more digits against an object. For example, without limitation, a squeeze sensor, a pressure sensitive wafer, or a force sensitive resistor, can be used as a digit pressure sensor according to the embodiments described herein.

As shown in FIG. 3, the servo motors can be externally powered by a power source 140. According to other embodiments, the motors may be powered by an internal power source situated within the prosthetic limb 60. In this way, the electronics of the prosthetic limb 60 can be made to be completely self-contained.

### Rotational Control—Second Movement

According to embodiments of the present disclosure, the input control device 10 can further effect control of the prosthetic limb 60 in a second movement. In such embodiments, the input control device 10 comprises a motion sensor 90 configured to sense tilting of the user's head and operable to convert the user's head tilting motion into a second electronic signal, wherein the second electronic signal is proportional to the user's head tilting motion. According to embodiments of the present disclosure, the motion sensor 90 can include two-axis or three-axis accelerometers. According to embodiments, the motion sensor 90, accelerometer, can be housed in the headset 40 in combination with the breath inlet 30. In this way, two movements can be controlled by the user through a single input control device 10 conveniently wearable on the user's head.

Referring to the electronic control circuitry shown in FIG. 3, the electronic signal output of the motion sensor 90 is converted into positioning data for the prosthetic limb 60. The typically analog output from the motion sensor 90 is converted to a digital format by an analog to digital converter (ADC). The converter may reside inside the accelerometer in certain embodiments. According to embodiments, an amplifier between the motion sensor 90 and the ADC may be used to condition the motion sensor 90 output signal. The data generated by the motion sensor, and any other amplifying circuit or data converter is transmitted to the processor 100 which generally then translates the electronic signal output into positional data for the wrist actuator 130, for example a servo motor, to move the prosthetic limb 60 in a second movement. In typical embodiments, some digital signal processing may take place in the processor to smooth jitter in the digit-actuators due to spurious head-shake and uneven data output from the accelerometer/motion sensor. In various embodiments, the data is transmitted serially via a standard data cable. The data cable may consist of simple stranded copper wire, it may also be a standard USB cable, although other compatible data cables are possible. Generally, other means of data communication are also possible. In some embodiments, it is possible that a fiber optic cable may be used for the data cable providing that the data output is converted from electrical data to optical data by an optical transmitter. In other embodiments, data may be communicated wirelessly providing that the data output is transmitted by a radio frequency (RF) transmitter.

According to preferred embodiments, the accelerometer 90 effects rotational movement of a wrist-actuator 130 to effect rotational movement of the wrist of a prosthetic arm 60 as illustrated in FIG. 2. In this way, the wrist of the prosthetic arm 60 can be controllably actuated to rotate in proportion and/or in the direction of the head tilting motion of the user.

### Operation

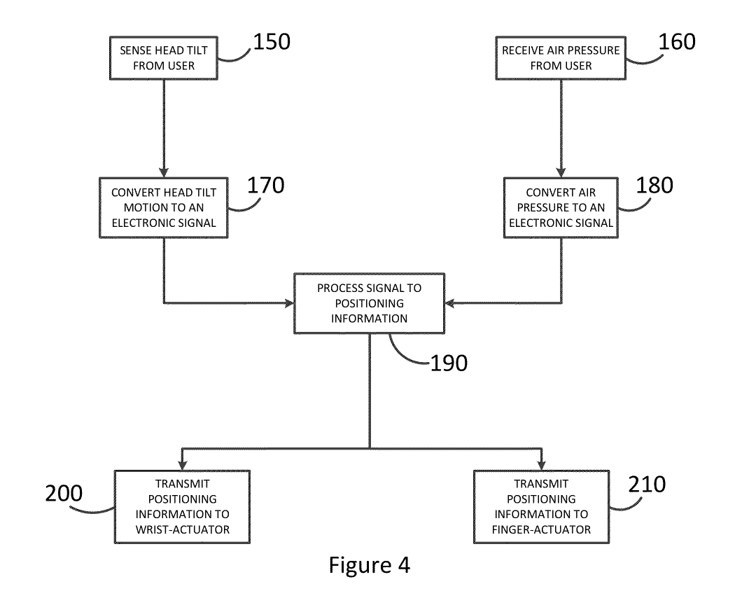

FIG. 4 illustrates the operation of various embodiments of the input control device 10 to effect two movements of a prosthetic arm 60, according to a preferred embodiment. According to embodiments, the first movement can be gripping control of the hand of a prosthetic arm 60. In operation, the gripping control can be effected by receiving air pressure from a user 160 and converting 180 the air pressure to a first electronic signal. The electronic signal that is generated is proportional to the air pressure exerted by the user. The first electronic signal is then processed 190 into first positional data for transmission 210 to a plurality of digit-actuators in the prosthetic limb to controllably actuate the respective digits, e.g., fingers, into the closed position.

According to further embodiments, the first movement can be expanded to provide the user with a relatively wide range of motions. In particular embodiments, the first movement can be customized with pre-programmed digit-actuator control impulses corresponding to pre-programmed hand positions, gestures, or movement combinations resulting from combinations of unique positive and/or negative breath pressure changes effecting control of the hand of a prosthetic arm 60. In operation, the pre-programmed gestures can be effected by receiving combinations of unique positive and/or negative breath pressure changes from a user 160 and converting 180 the air pressure to a first electronic signal. The electronic signal that is generated is activated by the air pressure changes exerted by the user. The first electronic signal is then processed 190 into first positional data for transmission 210 to a plurality of digit-actuators in the prosthetic limb to controllably actuate the respective digits, e.g., fingers, into a range of pre-programmed positions, gestures, or movement combinations, for example, individual finger movements such as to form the “peace sign”, pointing of various fingers, etc.

According to embodiments, the finger tips or pads of the prosthetic 60 can further include pressure sensors to allow the grip strength to be controlled. In such an embodiment, for example, a pattern of unique positive and/or negative breath pressure changes could be used to trigger hand closure until finger sensor limits are reached. In this way, data for haptic feedback could also be provided to the user to give the user the ability to “feel” the held object. According to embodiments, a proportionally vibrating motor or other active device can be used to provide such haptic feedback. Further embodiments may take advantage of cutting edge nerve-induction techniques for haptic feedback.

According to embodiments, the second movement can be wrist rotation of the prosthetic arm 60. In operation, the rotational movement can be effected by sensing 150 a tilting motion of the user's head and converting 170 the tilting motion into a second electronic signal. The second electronic signal that is generated is proportional to, and according to certain embodiments in the direction of, the user's head tilting motion. The second electronic signal is then processed 190 into second positional data and transmitted 200 to the wrist-actuator in the prosthetic limb. The wrist-actuator is thereby actuated to rotate in proportion to the head tilting motion of the user.

To gain a better understanding of the invention described herein, the following examples are set forth. It will be understood that these examples are intended to describe illustrative embodiments of the invention and are not intended to limit the scope of the invention in any way.

### EXAMPLES

### Example 1

### Prototype Breath, Head-Tilt Controlled Prosthetic Hand

A low-cost, open-source, 3D printed prosthetic hand prototype controlled by a breath-pressure sensor and dual axis accelerometer was developed. Finger movements and wrist rotation were made controllable by separate control systems. A breath-pressure sensor was used to control finger movement and a dual axis accelerometer implanted in a headset was used to control wrist rotation.

### Hand

Materials and Methods
3D printed PLA plastic hand, wrist and forearm (open source design by inMoov)
Various nails, screws, and bolts
Locktite Super Glue (gel and liquid)
Spiderwire 100 lb test braided fishing line
5—Hobby King HK-15298 90°, high current (14 lb torque) servo motors (4.8-7V)
1 Parallax standard 180° servo motor (4.8-6V)
1 USB cable
Hookup wire
4 in. small plastic fuel-line tubing
3/16 in. outer diameter flexible plastic tubing
3-way plastic tubing air splitter
Green Stuff modeling putty
1 Arduino Uno microprocessor
1 Arduino proto-shield
1 Freescale MP3V5010GP Pressure Sensor
6, 3-pin male servo headers
3 small cable binders
1 silicone oven mitt
Variable high amperage, power supply
5 VDC, 1000 mA USB power supply

The prosthetic hand prototype was constructed from purchased, modified, and 3D printed parts. A 3D printed PLA plastic hand, wrist and forearm (open source design by inMoov) was equipped with five fishing-line tendons (Spiderwire 100 lb test braided fishing line) actuated by five servo motors (Hobby King HK-15298 90°, high current (14 lb torque) servo motors (4.8-7V)) and controlled by a breath-pressure sensor (Freescale MP3V5010GP Pressure Sensor) to provide breath-controlled hand opening and closing.

The hand could be closed in proportion to the amount of negative breath-pressure (sucking) applied to the breath-pressure sensor. This sensor could control the five servomotors, which pull on the five fishing-line tendons to close the hand. When sucking was stopped and normal pressure returned to the sensor, the servos could pull the hand back open.

The Freescale MP3V5010GP pressure sensor used in the prototype had a sensitivity range of 0 to 1.4 psi (a normal human exhale range). The sensor had an element which output a voltage from 0.1 to 3.0 VDC in direct proportion to the exerted pressure. The sensor output serial values between 55 and 666 for positive breath pressure, and 55 and 5 for negative breath pressure. When testing the sensor, negative pressure seemed to be the most intuitive means to operate the finger servos.

Wrist rotation was made controllable by including a servo motor (Parallax standard 180° servo motor (4.8-6V)) in the wrist joint of the prototype prosthetic arm. The wrist joint was controlled by an accelerometer (Memsic 2125 dual axis accelerometer) placed in the earpiece of a headset. When the user tilts his head, the wrist joint turns proportionally in the direction of the tilt.

The Memsic 2125 accelerometer used in the prototype has a 100 Hz square wave output with a 50% duty cycle at 0 tilt. When the sensor is tilted, the ratio of the on cycle to the off cycle is changed in proportion to the motion of a heated gas bubble inside the accelerometer. This ratio is scaled in the processor according to the data provided in the Memsic data sheet and the output is then processed and mapped onto the wrist servo.

All of the electronics for the control software systems were designed to be located inside the hand so that an external CPU would not need to be relied on. The control software systems were customized for the Arduino microprocessor platform inside the hand. All control systems were located within the hand (e.g., microprocessor, servo motors, breath-pressure sensor, wiring, etc.) except for the accelerometer. To accomplish this, significant internal modifications were done to the lower wrist and upper forearm segments of the prosthetic arm.

### Control Headset

Materials and Methods
Standard voice headset
1 Memsic 2125 dual axis accelerometer
3 ft. of 3/16 in. outer diameter flexible plastic tubing
Jumbo flexible plastic drinking straw
4 ft. of 22 gauge 4-strand communication wire
5 female pin headers
heat-shrinkable tubing

The input control systems were made wearable by the user in a headset for easy access by the user. The microphone boom from a headset was adapted to hold the breath-pressure sensor tubing and the accelerometer was implanted in the earpiece after removing the speakers.

### Electronics and Software Systems

Based on information from the accelerometer and breath-pressure sensor data sheets, a circuit system was designed. FIG. 3 illustrates how each sensor was attached to the microprocessor, where each component received its power supply, and the data lines for the servomotors.

The hand/wrist control software combined code harvested from two different sensor development codes found in the Arduino IDE (Integrated Development Environment). The code for the breath sensor was a modified version of the flex-sensor servo control code. Accelerometer control of the wrist servo was a modified version of the Memsic data acquisition code also found in the Arduino IDE. These two codes were combined and modified to include a wrist servo, 5 finger-servos and two data processing algorithms. The data processing was important as it prevented the servos from excessive jittering. The code itself operated as follows: Data from the breath-pressure sensor was acquired by the program and stored in a memory buffer. After being filled to 10 places, the data was averaged, scaled and sent to the finger control servos. The memory buffer was continuously refilled with data so that new sensor input could be processed. Data from the Y-axis of the accelerometer was acquired by the program and also stored in a memory buffer. The X-axis data was not processed, as this axis was not used. Like the breath data, after the buffer was filled to 10 places, the data was averaged and scaled. To prevent additional jitters in the data, a cutoff range was established to prevent unwanted values from passing to the wrist servo. The scaled data was then sent to the wrist control servo. This memory buffer was also continuously refilled with data so that new sensor input could be processed.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

The embodiments of the invention in which an exclusive property or privilege is claimed are defined as follows:

1. An input control device for a power-driven prosthetic limb, comprising:
a breath inlet configured to receive air pressure exerted from a user's mouth to be converted into a first electronic signal, wherein the first electronic signal is proportional to the exerted pressure;
a motion sensor configured to sense tilting of the user's head and operable to convert the user's head tilting motion into a second electronic signal, wherein the second electronic signal is proportional to the user's head tilting motion; and
a processor in communication with the breath inlet and the motion sensor, the processor operable to convert the exerted air pressure into the first electronic signal and further process the first electronic signal into first positional data for transmission to a plurality of digit-actuators in the prosthetic limb, the processor further operable to process the second electronic signal into second positional data for transmission to a wrist-actuator in the prosthetic limb;
wherein the plurality of digit-actuators in the prosthetic limb can be controllably actuated in proportion to the air pressure exerted by the user, and the wrist-actuator can be controllably actuated to rotate in proportion to the head tilting motion of the user.

2. The input control device according to claim 1, wherein the breath inlet and the motion sensor are housed in a headset wearable by the user.

3. The input control device according to claim 1, wherein the motion sensor comprises an accelerometer.

4. The input control device according to claim 1, wherein the processor comprises a breath pressure sensor, an amplifier and an analog to digital converter (ADC).

5. The input control device according to claim 1, wherein the processor is in wireless communication with the breath inlet, the motion sensor, the plurality of digit-actuators, and the wrist-actuator.

6. The input control device according to claim 1, wherein the first and second positional data is serially transmitted from the processor.

7. The input control device according to claim 1, wherein the exerted air pressure is a combination of positive and negative air pressure created by the user exhaling or blowing and inhaling or sucking air into the breath inlet.

8. The input control device according to claim 4, wherein the breath pressure sensor can detect air pressures ranging from about 0 psi to about 1.4 psi.

9. The input control device according to claim 1, wherein the first electronic signal has a voltage of between about 0.1 VDC to about 3.0 VDC in direct proportion to the exerted air pressure.

10. The input control device according to claim 1, further comprising a feedback control loop for controlling an upper limit of actuating the plurality of digit-actuators.

11. The input control device according to claim 10, wherein the feedback control loop comprises one or more pressure sensors in each of the plurality of digits, each pressure sensor configured to sense pressure exerted by the respective digit against an object and operable to convert the exerted pressure into a feedback electronic signal proportional to the exerted pressure, wherein the feedback electronic signal is transmitted to the processor to halt actuation of the plurality of digit-actuators when the upper limit is reached.

12. A powered prosthetic limb, comprising:
a first input control comprising a breath inlet configured to receive air pressure exerted from a user's mouth to be converted into a first electronic signal, wherein the first electronic signal is proportional to the exerted pressure;
a second input control comprising a motion sensor configured to sense tilting of the user's head and operable to convert the user's head tilting motion into a second electronic signal, wherein the second electronic signal is proportional to the user's head tilting motion;
a prosthetic limb comprising a plurality of digit-actuators and a wrist-actuator, each digit-actuator attached to a respective artificial tendon or gear system, wherein the respective artificial tendon or gear system can be retracted or activated by the respective digit-actuator; and
a processor housed in the prosthetic limb in communication with the first and second input control, the processor operable to convert the exerted air pressure into the first electronic signal and further process the first electronic signal into first positional data for transmission to the plurality of digit-actuators in the prosthetic limb, the processor further operable to process the second electronic signal into second positional data for transmission to the wrist-actuator in the prosthetic limb;
wherein the plurality of digit-actuators in the prosthetic limb can be controllably actuated in proportion to the air pressure exerted by the user, and the wrist-actuator can be controllably actuated to rotate in proportion to the head tilting motion of the user.

13. The powered prosthetic limb according to claim 12, wherein the first positional data actuates the plurality of digit-actuators into a closed position.

14. The powered prosthetic limb according to claim 12, wherein the first positional data corresponds to customized pre-programmed digit-actuator control impulses to actuate pre-programmed movement combinations.

15. The prosthetic limb according to claim 12, wherein the prosthetic limb is an arm with a hand comprising finger-actuators, each finger-actuator attached to the respective artificial tendon or gear system, wherein the respective artificial tendon or gear system can be retracted or activated by the respective finger-actuator to a closed position.

16. A method of controlling a prosthetic limb, comprising:
receiving air pressure from a user and converting the air pressure to a first electronic signal, wherein the first electronic signal is proportional to the air pressure;
sensing a tilting motion of the user's head and converting the tilting motion into a second electronic signal, wherein the second electronic signal is proportional to the user's head tilting motion;
processing the first electronic signal into first positional data for a plurality of digit-actuators in the prosthetic limb and processing the second electronic signal into second positional data for a wrist-actuator in the prosthetic limb; and
transmitting the first positional data to the plurality of digit-actuators, and transmitting the second positional data to the wrist-actuator;
wherein the plurality of digit-actuators in the prosthetic limb can be controllably actuated in proportion to the air pressure exerted by the user, and the wrist-actuator can be controllably actuated to rotate in proportion to the head tilting motion of the user.

17. The method according to claim 16, further comprising:
sensing pressure exerted by one or more digits against an object and converting the exerted pressure into a feedback electronic signal proportional to the exerted pressure; and
transmitting the feedback electronic signal to the processor effecting an upper limit for actuation of the plurality of digit-actuators;
wherein actuation of the plurality of digit-actuators is halted when the upper limit is reached.

18. The method according to claim 17, wherein the processor compares the feedback electronic signal with preset pressure signal limits to effect the upper limit.

19. The method according to claim 17, wherein one or more digit pressure sensors is used to sense the pressure exerted by one or more digits against an object.

20. The method according to claim 19, wherein the one or more digit pressure sensors is a squeeze sensor, a pressure sensitive wafer, or a force sensitive resistor.
